(19) United States
(12) Patent Application Publication
(10) Pub. No.: US 2021/0388309 A1
(43) Pub. Date: Dec. 16, 2021

(54) PROCESSES FOR TREATMENT OF MICROBE SUSPENSIONS

(71) Applicant: Monsanto Technology LLC, St. Louis, MO (US)

(72) Inventors: Lorenzo AULISA, Chesterfield, MO (US); Mingya HUANG, Creve Coeur, MO (US); Hadi SHAMSIJAZEYI, St. Louis, MO (US); Hua WANG, Chesterfield, MO (US)

(21) Appl. No.: 17/291,058

(22) PCT Filed: Nov. 6, 2019
(86) PCT No.: PCT/US2019/060027

§ 371 Date: May 4, 2021

Related U.S. Application Data
(60) Provisional application No. 62/756,403, filed on Nov. 6, 2018.

(51) Int. Cl. C12N 1/02; C12N 1/14; C12N 1/20; C08L 31/06; C08L 97/02

(52) U.S. Cl. CPC C12N 1/02; C12N 1/14; C12N 2500/30; C08L 31/06; C08L 97/02; C12N 1/20

(57) ABSTRACT

Various processes for the treatment of microbe suspensions and associated compositions thereof are described.

### FIELD

Provided herein are various processes for the treatment of microbe suspensions and associated compositions thereof.

### BACKGROUND

Spores of some species of microbes can form relatively large aggregates, especially during or after fermentation. Large aggregates of microbe spores can potentially cause problems for microbial formulations, including those developed for seed treatments. For example, seed treatments containing large aggregates of spores can result in uneven or non-uniform coatings to be applied to the seeds. Mechanical methods to reduce the size of large aggregates of microbe spores can sometimes require high shear forces to be applied to the aggregates that can compromise the viability of the microbe spores. Accordingly, there remains a need for new processes that can effectively and efficiently reduce the size of microbe spores without significantly compromising the viability of the microbe spores.

### BRIEF SUMMARY

Various embodiments include processes for de-aggregating microbe spore aggregates. In some embodiments, these processes comprise mixing a polymeric additive with a first suspension comprising the microbe spore aggregates and one or more other components to form a second suspension comprising the polymeric additive, microbe spores and/or aggregates thereof, and the one or more other components, wherein the average particle size of the microbe spores and/or aggregates thereof in the second suspension is less than the average particle size of the microbe spore aggregates in the first suspension.

Other embodiments include processes for producing a suspension concentrate comprising microbe spores and/or aggregates thereof. In various embodiments, these processes comprise mixing a polymeric additive with a first suspension comprising the microbe spore aggregates and one or more other components to form a second suspension comprising the polymeric additive, microbe spores and/or aggregates thereof, and the one or more other components, wherein the average particle size of the microbe spores and/or aggregates thereof in the second suspension is less than the average particle size of the microbe spore aggregates in the first suspension. These processes further comprise separating at least a portion of the microbe spores and/or aggregates thereof from the one or more other components to form the suspension concentrate comprising the microbe spores and/or aggregates thereof

Further embodiments include various microbe compositions. For example, in some embodiments, the microbe composition comprises microbe spores and/or aggregates thereof and a polymeric additive, wherein at least one of the following conditions is satisfied:

(a) the average particle size of the microbe spores and/or aggregates thereof in the composition is less than about 50 μm, less than about 40 μm, less than about 30 μm, less than about 20 μm, less than about 10 μm, less than about 5 μm, or less than about 2 μm;

(b) the composition further comprises one or more other components;

(c) the concentration of the polymeric additive in the composition is at least about 2 wt. %, at least about 2.5 wt. %, at least about 3 wt. %, at least about 3.5 wt. %, at least about 4 wt. %, at least about 4.25 wt. %, at least about 4.5 wt. %, at least about 5 wt. %, or at least about 6 wt. %; and/or

(d) the ratio of the microbe spores in CFUs to mass of the polymeric additive in grams in the composition is at least about 2×10⁵:1, at least about 2×10⁶:1, at least about 2×10⁷:1, at least about 2×10⁸:1, at least about 2×10⁹:1, or at least about 2×10¹⁰:1.

Other objects and features will be in part apparent and in part pointed out hereinafter.

### BRIEF DESCRIPTION OF THE DRAWINGS

FIG. 1 shows an exemplary particle size reduction that is consistent with the methods of the present invention.

FIG. 2 depicts the particle size distribution for microbe suspensions containing SOKALAN CP9, POLYFON O or VULTAMOL NH 7519.

FIG. 3 depicts the particle size distribution at time 0 h for microbe suspensions containing POLYFON O at concentrations ranging from 2.0 wt % to 6.0 wt %.

FIG. 4 depicts the particle size distribution at time 24 h for microbe suspensions containing POLYFON O at concentrations ranging from 2.0 wt % to 6.0 wt %.

FIG. 5 depicts the particle size distribution at time 0 h for microbe suspensions containing 4.0 wt % sodium lignosulfonate.

FIG. 6 depicts the particle size distribution at time 20 h for microbe suspensions containing 4.0 wt % sodium lignosulfonate.

FIG. 7 depicts the particle size distribution at time 0 h for microbe suspensions containing various polymeric additives.

FIG. 8 depicts the particle size distribution at time 24 h for microbe suspensions containing various polymeric additives.

FIG. 9 depicts the particle size distribution at time 44 h for microbe suspensions to examine the effect of the timing of addition of the polymeric additive in relation to centrifugation.

### DETAILED DESCRIPTION

Various processes for the treatment of microbe suspensions and associated compositions thereof are described herein. For example, some embodiments include processes for de-aggregating microbe spore aggregates. In various embodiments, processes for de-aggregating microbe spore aggregates comprise mixing a polymeric additive with a first suspension comprising the microbe spore aggregates and one or more other components to form a second suspension comprising the polymeric additive, microbe spores and/or aggregates thereof, and the one or more other components, wherein the average particle size of the microbe spores and/or aggregates thereof in the second suspension is less than the average particle size of the microbe spore aggregates in the first suspension.

Some embodiments include processes for producing a suspension concentrate comprising microbe spores and/or aggregates thereof. In various embodiments, these processes comprise mixing a polymeric additive with a first suspension comprising the microbe spore aggregates and one or more other components to form a second suspension comprising the polymeric additive, microbe spores and/or aggregates thereof, and the one or more other components, wherein the average particle size of the microbe spores and/or aggregates thereof in the second suspension is less than the average particle size of the microbe spore aggregates in the first suspension. These processes further comprise separating at least a portion of the microbe spores and/or aggregates thereof from the one or more other components to form the suspension concentrate comprising the microbe spores and/or aggregates thereof.

Further embodiments include various microbe compositions (e.g., microbe suspensions and suspension concentrates). For example, in some embodiments, the microbe composition comprises microbe spores and/or aggregates thereof and a polymeric additive.

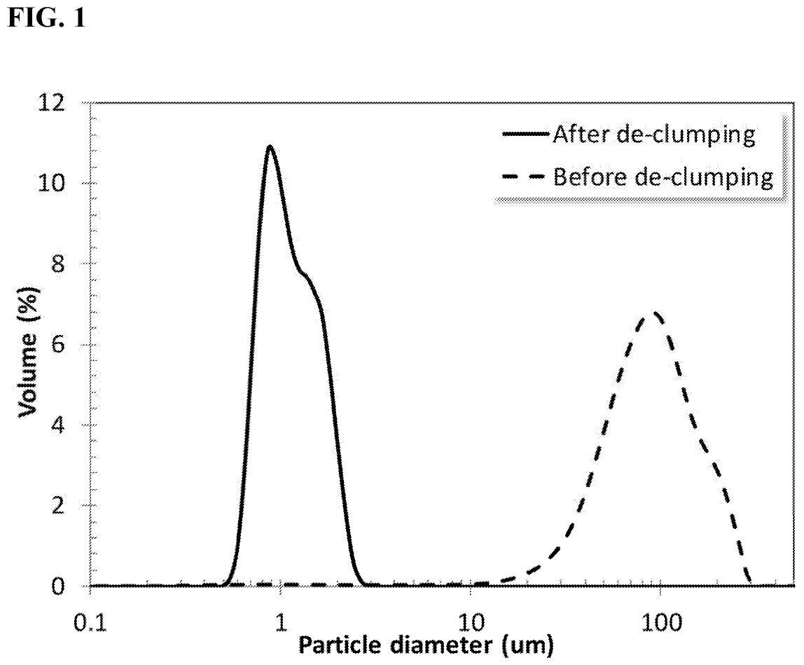

As noted, spores of some species of microbes can form relatively large aggregates, particularly during or subsequent to fermentation. Applicants have discovered that these relatively large aggregates of microbe spores can be de-aggregated according to the processes described herein. In particular, it has been found that various polymeric additives can break down these aggregates thereby forming improved suspensions comprising individual microbe spores and/or aggregates thereof having substantially reduced particle size (e.g., an average particle size that is reduced by a factor of at least about 5, at least about 10, at least about 25, at least about 50, or even at least about 100). FIG. 1 shows an exemplary particle size reduction that is consistent with the methods of the present invention. Suspensions comprising individual microbe spores and/or relatively small aggregates thereof are beneficial for use in applications that require relatively stable, homogeneous suspensions, such as seed treatment formulations.

The microbe spore aggregates comprise microbe spores. The microbe spores can be fungus spores, bacteria spores, or a combination thereof. Generally, the microbe spores comprise fungus spores and/or bacteria spores that are particularly susceptible to aggregation during or after fermentation. For example, in various embodiments, the microbe spores can comprise bacteria spores from at least one genus selected from the group consisting of Actinomycetes, Azotobacter, Bacillus, Brevibacillus, Burkholderia, Paenibacillus, Pasteuria, Photorhabdus, Phyllobacterium, Xenorhabdus, or combinations thereof. In some embodiments, the microbe spores comprise bacteria spores from at least one species selected from the group consisting of Bacillus amyloliquefaciens, Bacillus cereus, Bacillus firmus, Bacillus lichenformis, Bacillus psychrosaccharolyticus, Bacillus pumilus, Bacillus sphaericus, Bacillus subtilis, Bacillus thuringiensis, Pasteuria penetrans, Pasteuria usgae, and combinations thereof. In certain embodiments, the microbe spores comprise bacteria spores of Bacillus psychrosaccharolyticus.

In further embodiments, the microbe spores comprise fungus spores from at least one genus selected from the group consisting of Alternaria, Ampelomyces, Aspergillus, Aureobasidium, Beauveria, Colletotrichum, Coniothyrium, Gliocladium, Metarhizium, Muscodor, Paecilomyces, Trichoderma, Typhula, Ulocladium, Verticillium, and combinations thereof. For example, the microbe spores can comprise fungus spores from at least one species selected from the group consisting of Beauveria bassiana, Coniothyrium minitans, Gliocladium vixens, Muscodor albus, Paecilomyces lilacinus, Trichoderma polysporum, and combinations thereof.

Typically, the microbe spore aggregates are suspended in liquid such as water (e.g., a first suspension comprising the microbe spore aggregates). In some embodiments, the processes can be used to treat suspensions having a wide range of microbe spore concentrations. In some embodiments, the concentration of microbe spores in the first suspension, second suspension and/or various microbe compositions described herein is at least about 1×10⁵ colony forming units (CFUs), 1×10⁶ CFUs, at least about 1×10⁷ CFUs, at least about 1×10⁸ CFUs, at least about 1×10⁹ CFUs, at least about 1×10¹⁰ CFUs, or at least about 1×10¹¹ CFUs. For example, the concentration of microbe spores in the first suspension, second suspension and/or various microbe compositions described herein can be from about 1×10⁵ CFUs to about 1×10¹¹ CFUs, from about 1×10⁶ CFUs to about 1×10¹¹ CFUs, from about 1×10⁷ CFUs to about 1×10¹¹ CFUs, from about 1×10⁸ CFUs to about 1×10¹¹ CFUs, or from about 1×10⁹ CFUs to 1×10¹¹ CFUs.

This first suspension and/or various microbe compositions described herein can comprise one or more other components (e.g., in addition to solvent/water), such as one or more microbe nutrients and preservatives. Microbe nutrients can include, for example, a carbon source such as various sugars (e.g., glucose), sugar alcohols, other carbohydrates, carbohydrate derivatives (e.g., glucosamine), and organic acids and salts thereof (e.g., sodium citrate and potassium gluconate), a nitrogen source such ammonia or nitrate salts, a phosphorous source such as various phosphates, amino acids (e.g., L-glutamic acid, L-leucine, L-valine, L-threonine, L-methionine, and L-histidine), yeast extract, and sources of various metals such as sodium (e.g., Na₂SO₄), potassium, calcium (e.g., CaCl₂ or Ca(NO₃)₂), magnesium (e.g., MgCl₂ or MgSO₄), iron (e.g., FeCl₂ or FeSO₄), zinc (e.g., ZnCl₂), manganese (e.g., MnSO₄), and cobalt (e.g., CoCl₂). In certain embodiments, the first suspension and/or various microbe compositions described herein comprises glucose at a concentration of at least about 5 mM, at least about 10 mM, or at least about 20 mM.

In various embodiments, the first suspension and/or various microbe compositions described herein comprises a fermentate (i.e., a crude suspension obtained from the fermentation broth of the microbe).

As noted, the average particle size of the microbe spore aggregates can be relatively large. In various embodiments, the average particle size of the microbe spore aggregates in the first suspension is at least about 50 μm, at least about 60 μm, at least about 70 μm, at least about 80 μm, at least about 90 μm, or at least about 100 μm. For example, in some embodiments, the average particle size of the microbe spore aggregates in the first suspension is from about 50 μm to about 500 μm, from about 60 μm to about 500 μm, from about 70 μm to about 500 μm, from about 80 μm to about 500 μm, from about 90 μm to about 500 μm, from about 100 μm to about 500 μm, from about 50 μm to about 400 μm, from about 60 μm to about 400 μm, from about 70 μm to about 400 μm, from about 80 μm to about 400 μm, from about 90 μm to about 400 μm, from about 100 μm to about 400 μm, from about 50 μm to about 300 μm, from about 60 μm to about 300 μm, from about 70 μm to about 300 μm, from about 80 μm to about 300 μm, from about 90 μm to about 300 μm, from about 100 μm to about 300 μm, from 50 μm to about 200 μm, from about 60 μm to about 200 μm, from about 70 μm to about 200 μm, from about 80 μm to about 200 μm, from about 90 μm to about 200 μm, from about 100 μm to about 200 μm, from about 50 μm to about 100 μm, from about 60 μm to about 100 μm, from about 70 μm to about 100 from about 80 μm to about 100 μm, or from about 90 μm to about 100 μm. Also, in various embodiments, at least about 80 vol. %, at least about 85 vol. %, at least about 90 vol. %, at least about 95 vol. %, or at least about 97 vol. % of the microbe spore aggregates in the first suspension have a particle size greater than about 10 μm, greater than about 25 μm, greater than about 50 μm, greater than about 75 μm, or greater than about 100 μm. The average particle size and particle size distributions of the microbe spores and microbe spore aggregates can be determined by measuring the particle size of a representative sample of a suspension as described herein with a laser light scattering particle size analyzer known to those skilled in the art. One example of a particle size analyzer is a Beckman Coulter LS Particle Size Analyzer.

As noted, in some embodiments, the processes described herein comprise mixing a polymeric additive with microbe spore aggregates (e.g., mixing a polymeric additive with a first suspension comprising the microbe spore aggregates). Also, various microbe compositions described herein comprise a polymeric additive. In various embodiments, the polymeric additive comprises a lignosulfonate. Lignosulfonates include various lignosulfonate salts such as sodium lignosulfonates, magnesium lignosulfonates, ammonium lignosulfonates, potassium lignosulfonates, calcium lignosulfonates, and combination thereof. In some embodiments, the polymeric additive comprises a sodium lignosulfonate.

In various embodiments, the average molecular weight of the lignosulfonate is at least about 1,000 Da, at least about 2,000 Da, or at least about 2,500 Da. For example, the average molecular weight of the lignosulfonate can be from about 1,000 Da to about 75,000 Da, from about 1,000 Da to about 50,000 Da, from about 1,000 Da to about 20,000 Da, from about 2,000 Da to about 20,000 Da, from about 2,000 Da to about 15,000 Da, from about 2,000 Da to about 10,000 Da, from about 2,000 Da to about 5,000 Da, from about 2,000 Da to about 4,000 Da, or from about 2,500 Da to about 4,000 Da.

Lignosulfonates can be characterized in part by the degree of sulfonation of the polymer molecule. For example, in some embodiments, the lignosulfonate has a degree of sulfonation that is from about 0.3 moles/kg to about 4 moles/kg, from about 0.5 moles/kg to about 4 moles/kg, from about 0.5 moles/kg to about 3.5 moles/kg, from about 1 moles/kg to about 3.5 moles/kg, from about 1.2 moles/kg to about 3.3 moles/kg, or from about 1.2 moles/kg to about 2 moles/kg. Lignosulfonates can also be characterized in part by content of organic sulfur. In various embodiments, the organic sulfur content of the lignosulfonate is from about 0.5 wt. % to about 20 wt. %, from about 1 wt. % to about 18 wt. %, from about 1 wt. % to about 16 wt. %, from about 2 wt. % to about 16 wt. %, from about 2 wt. % to about 10 wt. %, or from about 4 wt. % to about 10 wt. %.

The sulfonic acid group of the lignosulfonate can be present at different locations on the polymer molecule. For example, the sulfonic acid group can be located on an aliphatic side chain and/or on an aromatic nucleus.

Various commercially available lignosulfonates include POLYFON F, POLYFON H, POLYFON O, POLYFON T, REAX 83A, REAX 105M, and REAX 907, available from Ingevity. Other lignosulfonates include BORRESPERSE NA, MARASPERSE AG, MARASPERSE N-22, MARASPERSE CBOS-4, UFOXANE 3A, and ULTRAZINE NA, available from Borregaard Lignotech.

In further embodiments, the polymeric additive comprises a maleic acid olefin copolymer. Suitable maleic acid/olefin polymers may comprise, for example, diisobutene, acrylic acid, or olefin copolymers. Non-limiting examples of commercially available maleic acid/olefin polymers include, for example, SOKALAN CP 9 and SOKALAN CP 5 available from BASF and AGRIMER VEMA H-2200L available from Ashland.

In various embodiments, the concentration of the polymeric additive in the second suspension and/or various microbe compositions described herein is at least about 2 wt. %, at least about 2.5 wt. %, at least about 3 wt. %, at least about 3.5 wt. %, at least about 4 wt. %, at least about 4.25 wt. %, at least about 4.5 wt. %, at least about 5 wt. %, or at least about 6 wt. %. For example, the concentration of the polymeric additive in the second suspension and/or various microbe compositions described herein is from about 2 wt. % to about 50 wt. %, from about 5 wt. % to about 50 wt. %, from about 10 wt. % to about 50 wt. %, from about 2 wt. % to about 40 wt. %, from about 5 wt. % to about 40 wt. %, from about 10 wt. % to about 40 wt. %, from about 2 wt. % to about 30 wt. %, from about 5 wt. % to about 30 wt. %, from about 10 wt. % to about 30 wt. %, from about 2 wt. % to about 20 wt. %, from about 5 wt. % to about 20 wt. %, from about 10 wt. % to about 20 wt. %, from about 2 wt. % to about 10 wt. %, from about 2.5 wt. % to about 8 wt. %, from about 3 wt. % to about 8 wt. %, from about 3.5 wt. % to about 8 wt. %, from about 4 wt. % to about 8 wt. %, from about 4 wt. % to about 6 wt. %, from about 4.25 wt. % to about 8 wt. %, or from about 4.25 wt. % to about 6 wt. %.

In some embodiments, the ratio of the microbe spores in CFUs to mass of the polymeric additive in grams in the second suspension and/or various microbe compositions described herein is at least about 2×10⁵:1, at least about 2×10⁶:1, at least about 2×10⁷:1, at least about 2×10⁸:1, at least about 2×10⁹:1, or at least about 2×10¹⁰:1. In certain embodiments, the ratio of the microbe spores in CFUs to mass of the polymeric additive in grams in the second suspension and/or various microbe compositions described herein is from about 2×10⁵:1 to about 5×10¹²:1, from about 2×10⁵:1 to about 5×10″:1, from about 2×10⁵:1 to about 5×10¹⁰:1, from about 2×10⁵:1 to about 5×10⁹:1, from about 2×10⁶:1 to about 5×10¹²:1, from about 2×10⁶:1 to about 5×10¹¹:1, from about 2×10⁶:1 to about 5×10¹⁰:1, from about 2×10⁶:1 to about 5×10⁹:1, from about 2×10⁷:1 to about 5×10¹¹:1, from about 2×10⁷:1 to about 5×10¹⁰:1, from about 2×10⁷:1 to about 5×10⁹:1, from about 2×10⁸:1 to about 5×10¹¹:1, from about 2×10⁸:1 to about 5×10¹⁰:1, from about 2×10⁸:1 to about 5×10⁹:1, from about 2×10⁹:1 to about 5×10¹¹:1, or from about 2×10⁹:1 to about 5×10¹⁰:1.

Generally, the average particle size of the microbe spores and/or aggregates thereof in the second suspension is less than the average particle size of the microbe spore aggregates in the first suspension. For example, the average particle size of the microbe spores and/or aggregates thereof in the second suspension and/or various microbe compositions described herein is no greater than about 50 μm, no greater than about 40 μm, no greater than about 30 μm, no greater than about 20 μm, no greater than about 10 μm, no greater than about 5 μm, or no greater than about 2 μm. In some embodiments, the average particle size of the microbe spores and/or aggregates thereof in the second suspension and/or various microbe compositions described herein is from about 0.5 μm to about 40 μm, from about 0.5 μm to about 30 μm, from about 0.5 μm to about 20 μm, from about 0.5 μm to about 10 μm, from about 0.5 p.m to about 5 μm, from about 0.5 μm to about 2 μm, from about 1 μm to about 40 μm, from about 1 μm to about 30 from about 1 μm to about 20 μm, from about 1 μm to about 10 μm, from about 1 μm to about 5 μm, from about 1 μm to about 2 μm, from about 1.5 μm to about 40 μm, from about 1.5 μm to about 30 μm, from about 1.5 μm to about 20 μm, from about 1.5 μm to about 10 μm, from about 1.5 μm to about 5 μm, from about 1.5 μm to about 2 μm, from about 2 μm to about 40 μm, from about 2 μm to about 30 μm, from about 2 μm to about 20 μm, from about 1 μm to about 10 or from about 2 μm to about 5 μm. In some embodiments, at least about 80 vol. %, at least about 85 vol. %, at least about 90 vol. %, at least about 95 vol. %, or at least about 97 vol. % of the microbe spores and/or aggregates thereof in the second suspension and/or various microbe compositions described herein have a particle size of less than about 10 μ.m or less than about 5 μm. In certain embodiments, at least about 80 vol. %, at least about 85 vol. %, at least about 90 vol. %, at least about 95 vol. %, or at least about 97 vol. % of the microbe spores and/or aggregates thereof in the second suspension and/or various microbe compositions described herein have a particle size between about 0.1 μm to about 10 μm or between about 0.5 μm to about 5 μm.

The addition of the polymeric additive may affect the pH of the suspension and/microbe composition (e.g., increase the pH). Accordingly, in various embodiments, the pH of the second suspension and/or various microbe compositions described herein is no greater than about 13, no greater than about 12.5, no greater than about 12, no greater than about 11.5, no greater than about 11, no greater than about 10.5, no greater than about 10, no greater than about 9.5, no greater than about 8.5, or no greater than about 8. For example, the pH of the second suspension and/or various microbe compositions described herein can be from about 7 to about 13, from about 7 to about 12.5, from about 7 to about 12, from about 7 to about 11.5, from about 7 to about 11, from about 7 to about 10.5, from about 7 to about 10, from about 7 to about 9.5, from about 7 to about 9, from about 7 to about 8.5, from about 7 to about 8, from about 8 to about 13, from about 8 to about 12.5, from about 8 to about 12, from about 8 to about 11.5, from about 8 to about 11, from about 8 to about 10.5, from about 8 to about 10, from about 8 to about 9.5, from about 8 to about 9, from about 8 to about 8.5, about 9 to about 13, from about 9 to about 12.5, from about 9 to about 12, from about 9 to about 11.5, from about 9 to about 11, from about 9 to about 10.5, from about 9 to about 10, from about 9 to about 9.5, about 10 to about 13, from about 10 to about 12.5, from about 10 to about 12, from about 10 to about 11.5, from about 10 to about 11, or from about 10 to about 10.5.

As noted, in some embodiments, the processes are directed to producing a suspension concentrate comprising microbe spores and/or aggregates thereof. These processes include the step of separating at least a portion of the microbe spores and/or aggregates thereof from the one or more other components to form the suspension concentrate comprising the microbe spores and/or aggregates thereof. In these processes, the concentration of the microbe spores and/or aggregates thereof in the suspension concentrate is typically greater than the concentration of the microbe spores and/or aggregates thereof in the second suspension. Also, the concentration of the one or more other components (e.g., microbe nutrients) and/or solvent (e.g., water) in the suspension concentrate is typically less than the concentration of these components in the second suspension. In some embodiments, separating at least a portion of the microbe spores and/or aggregates thereof comprises centrifugation and/or filtration.

Some embodiments are directed to processes which include various combinations of features described herein. For example, various processes for de-aggregating microbe spore aggregates comprise mixing a polymeric additive with a first suspension comprising the microbe spore aggregates and one or more other components to form a second suspension comprising the polymeric additive, microbe spores and/or aggregates thereof, and the one or more other components, wherein the average particle size of the microbe spores and/or aggregates thereof in the second suspension is less than the average particle size of the microbe spore aggregates in the first suspension and wherein at least one of the following conditions is satisfied:

(a) the average particle size of the microbe spore aggregates in the first suspension is at least about 50 μm, at least about 60 μm, at least about 70 μm, at least about 80 μm, at least about 90 μm, at least about 100 μm, or at least about 200 μm;

(b) the average particle size of the microbe spores and/or aggregates thereof in the second suspension is less than about 50 less than about 40 μm, less than about 30 μm, less than about 20 μm, less than about 10 μm, less than about 5 μm, or less than about 2 μm;

(c) the one or more other components comprises one or more microbe nutrients;

(d) the concentration of the polymeric additive in the second suspension is at least about 2 wt. %, at least about 2.5 wt. %, at least about 3 wt. %, at least about 3.5 wt. %, at least about 4 wt. %, at least about 4.25 wt. %, at least about 4.5 wt. %, at least about 5 wt. %, or at least about 6 wt. %; and/or

(e) the ratio of the microbe spores in CFUs to mass of the polymeric additive in grams in the second suspension is at least about 2×10⁵:1, at least about 2×10⁶:1, at least about 2×10⁷:1, at least about 2×10⁸:1, at least about 2×10⁹:1, or at least about 2×10¹⁰:1.

Various processes for producing a suspension concentrate comprising microbe spores and/or aggregates thereof comprise mixing a polymeric additive with a first suspension comprising the microbe spore aggregates and one or more other components to form a second suspension comprising the polymeric additive, microbe spores and/or aggregates thereof, and the one or more other components, wherein the average particle size of the microbe spores and/or aggregates thereof in the second suspension is less than the average particle size of the microbe spore aggregates in the first suspension; and separating at least a portion of the microbe spores and/or aggregates thereof from the one or more other components to form the suspension concentrate comprising the microbe spores and/or aggregates thereof. In various embodiments, at least one of the following conditions is satisfied:

(a) the average particle size of the microbe spore aggregates in the first suspension is at least about 50 μm, at least about 60 μm, at least about 70 μm, at least about 80 μm, at least about 90 μm, at least about 100 μm, or at least about 200 μm;

(b) the average particle size of the microbe spores and/or aggregates thereof in the second suspension is less than about 50 less than about 40 μm, less than about 30 μm, less than about 20 μm, less than about 10 μm, less than about 5 μm, or less than about 2 μm;

(c) the one or more other components comprises one or more microbe nutrients; (d) the concentration of the polymeric additive in the second suspension is at least about 2 wt. %, at least about 2.5 wt. %, at least about 3 wt. %, at least about 3.5 wt. %, at least about 4 wt. %, at least about 4.25 wt. %, at least about 4.5 wt. %, at least about 5 wt. %, or at least about 6 wt. %; and/or

(e) the ratio of the microbe spores in CFUs to mass of the polymeric additive in grams in the second suspension is at least about 2×10⁵:1, at least about 2×10⁶:1, at least about 2×10⁷:1, at least about 2×10⁸:1, at least about 2×10⁹:1, or at least about 2×10¹⁰:1.

Some embodiments are directed to microbe compositions which include various combinations of features described herein. For example, in certain embodiments, the microbe composition comprises microbe spores and/or aggregates thereof and a polymeric additive, wherein at least one of the following conditions is satisfied:

(a) the average particle size of the microbe spores and/or aggregates thereof in the composition is less than about 50 μm, less than about 40 μm, less than about 30 μm, less than about 20 μm, less than about 10 μm, less than about 5 μm, or less than about 2 μm;

(b) the composition further comprises one or more other components;

(c) the concentration of the polymeric additive in the composition is at least about 2 wt. %, at least about 2.5 wt. %, at least about 3 wt. %, at least about 3.5 wt. %, at least about 4 wt. %, at least about 4.25 wt. %, at least about 4.5 wt. %, at least about 5 wt. %, or at least about 6 wt. %; and/or

(d) the ratio of the microbe spores in CFUs to mass of the polymeric additive in grams in the composition is at least about 2×10⁵:1, at least about 2×10⁶:1, at least about 2×10⁷:1, at least about 2×10⁸:1, at least about 2×10⁹:1, or at least about 2×10¹⁰:1.

Having described the invention in detail, it will be apparent that modifications and variations are possible without departing from the scope of the invention defined in the appended claims.

### EXAMPLES

The following non-limiting examples are provided to further illustrate the present invention.

### Example 1

The compositions of Example 1 were prepared to evaluate the comparative reduction in particle size of microbial spore aggregates in fermentate (fermentation broth after completion of fermentation) by utilizing polymeric additives.

After fermentation was complete, individual polymeric additives (solid) were added to the fermentation broth (˜20 g) containing Bacillus psychrosaccharolyticus spores to a concentration of 2 wt. %. The compositions had an approximate microbe titer of 1×10⁸ to 1×10⁹ CFUs. To prevent additional microbial growth, a preservative (e.g. PROXEL GXL) was added prior to the addition of the polymeric additives. The samples were mixed for about 10 min using an EBERBACH Fixed Speed Reciprocal Shaker (Model E6010) on HIGH speed to allow for dissolution of the polymeric additives and complete mixing of components. After the mixing was completed the solids were allowed to settle and the samples were visually inspected at time 0 and after 68 h. The compositions containing larger aggregates settled more quickly than those containing smaller ones.

Table 1 reports compositions comprising commercially available polymeric additives and results from visual inspection.

TABLE 1

| | | Visual Settling |
|---|---|---|
| Polymeric Additive | Polymeric Additive Type | Observed |
| ATLAS G-5000 | Polyalkylene oxide block copolymer | Significant |
| ATLAS G-5002L | Butyl block copolymer | Significant |
| ATLOX 4894 | Nonionic surfactant blend | Significant |
| ATLOX 4913 | Acrylic copolymer | Significant |
| ATLOX 4915 | Polymeric amphoteric | Significant |
| BORRESPERSE NA | Sodium lignosulfonate | Significant |
| PLURONIC L64 | EO/PO block copolymer (40% EO, | Significant |
| | 2900 MW) | |
| PLURONIC P104 | EO/PO block copolymer (40% EO, | Significant |
| | 5900 MW) | |
| PLURONIC P105 | EO/PO block copolymer (50% EO, | Significant |
| | 6500 MW) | |
| POLYFON O | Sodium lignosulfonate | Minor |
| SOKOLAN CP9 | Maleic acid/olefin co-polymer | Minor |
| T-MAZ 85 K | Ethoxylated sorbitan monostearate (POE20) | Significant |
| TRYFAC 5560A | Tridecyl alcohol phosphate ester (POE6) acid form | Significant |
| VULTAMOL NH 7519 | Sodium naphthalene sulfonate condensate | Significant |
| VULTAMOL DN | Sodium phenol sulfonate condensate | Significant |

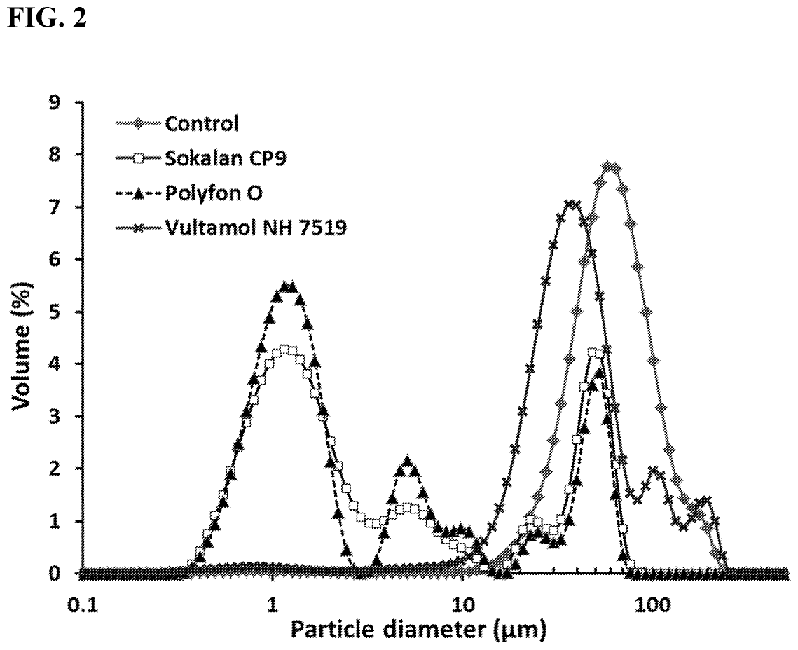

Within 2 weeks, particle size analysis, using a BECKMAN COULTER LS 13 320 laser diffraction particle size analyzer, was performed on three samples containing SOKALAN CP9, POLYFON O or VULTAMOL NH 7519 in addition to a control sample without a polymeric additive. Results from the particle size analysis are shown in FIG. 2. Additionally, the pH of the three polymeric additive-containing compositions was measured and determined to be 11.6, 9.5, and 8.9, respectively.

### Example 2

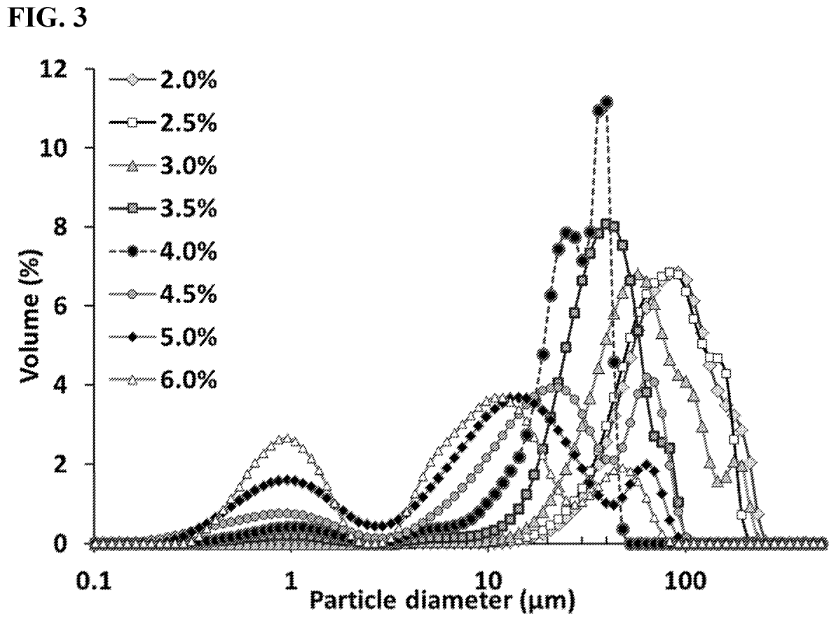

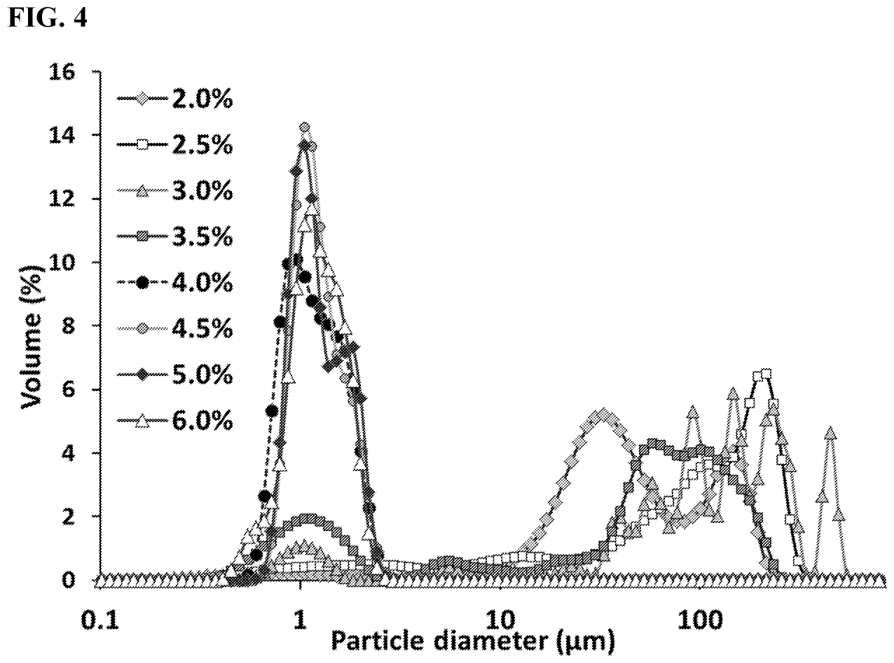

Compositions containing the polymeric additive POLYFON O were further evaluated to determine the optimal concentration range for reducing microbial aggregates. Similar to the procedure described above, POLYFON O was added to microbial fermentates (˜30 g) for final polymeric additive concentrations of 2.0, 2.5, 3.0, 3.5, 4.0, 4.5, 5.0, and 6.0 wt %. As described above, particle size analysis was performed on each sample at 0 h and 24 h. Results from the particle size analysis are shown in FIG. 3 and FIG. 4.

### Example 3

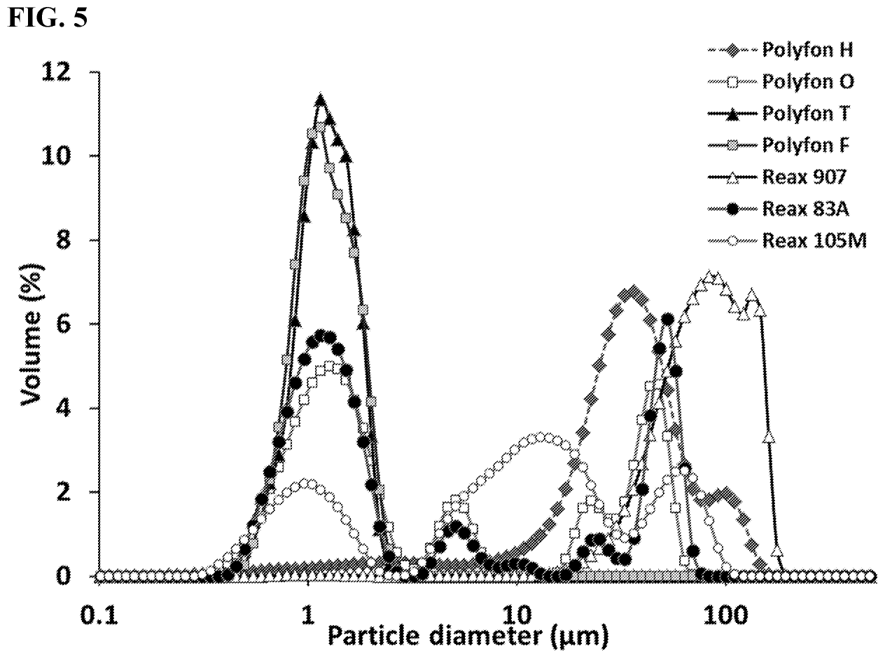

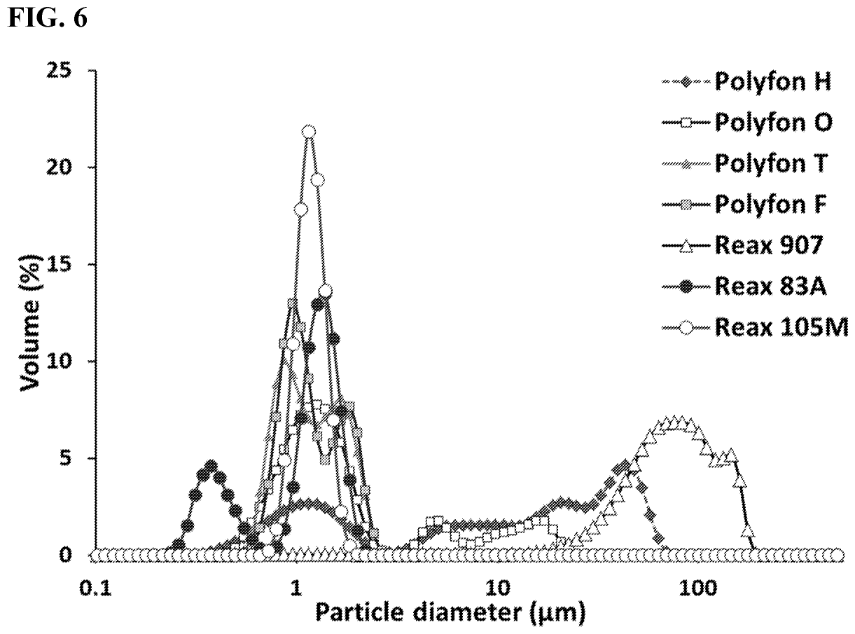

Compositions containing additional sodium lignosulfonates commercially available from Ingevity were evaluated to determine their effect on reducing microbial aggregates. Similar to the procedures described above, sodium lignosulfonates were individually added to microbial fermentates (30 g) for a final concentration of 4.0 wt % in each composition. Subsequent particle size analysis was performed on each sample at time 0 h and 20 h. Results from the particle size analysis are shown in FIG. 5 and FIG. 6.

TABLE 2

| | | Calc Degree | | Na₂SO₃ |
|---|---|---|---|---|
| Polymeric | | of Sulfonation | Site of Sulfonic | Content |
| Additive | Avg MW | (Moles/kg) | Acid Group | (%) |
| POLYFON F | 2900 | 3.3 | Aliphatic side chain | 16 |
| POLYFON H | 4300 | 0.7 | Aliphatic side chain | 3.5 |
| POLYFON O | 2400 | 1.2 | Aliphatic side chain | 5 |
| POLYFON T | 2900 | 2.0 | Aliphatic side chain | 9.5 |
| REAX 83A | 9000 | 1.8 | Aromatic nucleus | 2.3 |
| REAX 105M | 2000 | 3.4 | hybrid | 1 |
| REAX 907 | 13400 | 1.2 | Aromatic nucleus | 0.5 |

### Example 4

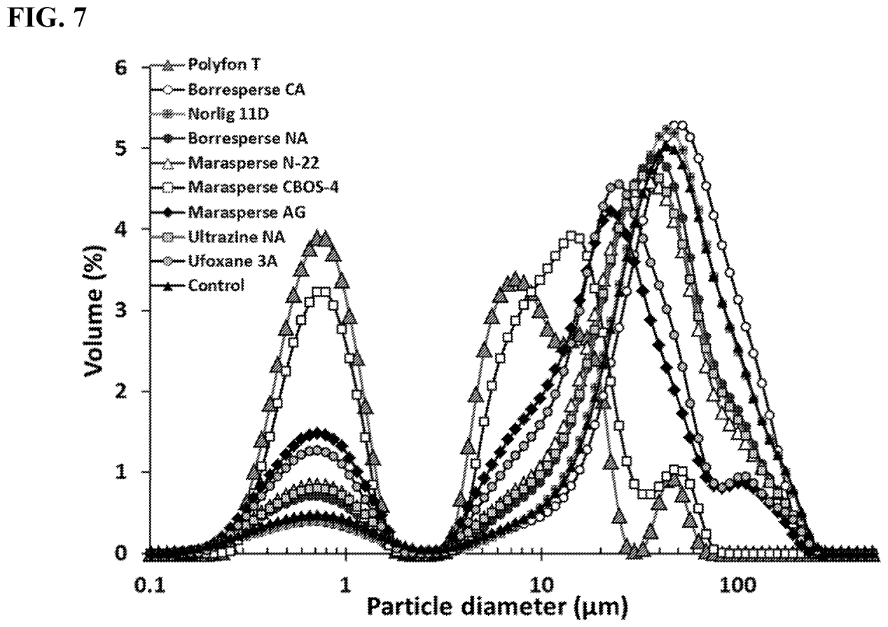

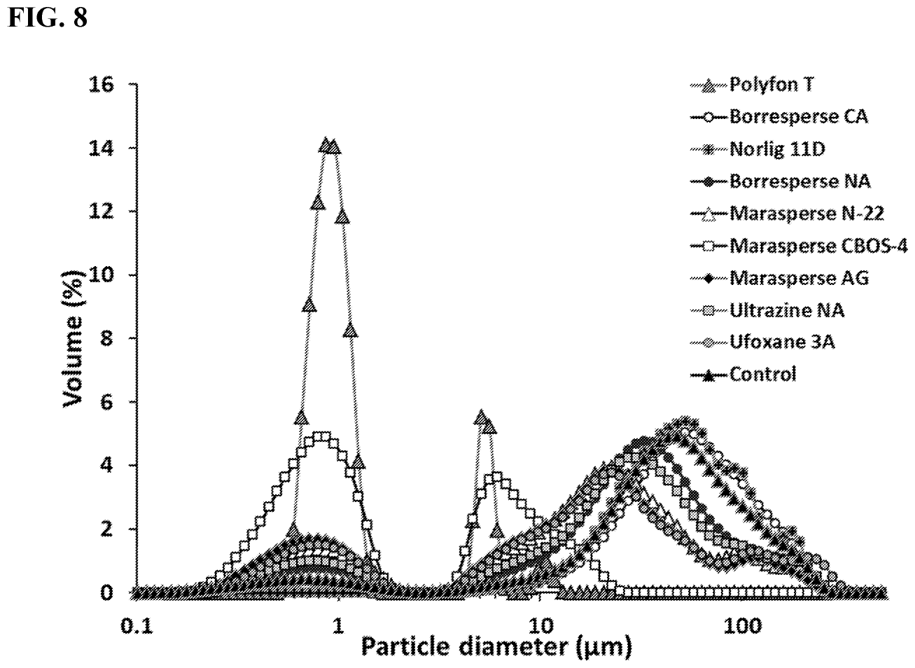

Compositions containing BORREGAARD LIGNOTECH polymeric additives commercially available from Borregaard were evaluated to determine their effect on reducing microbial aggregates. POLYFON T was used as an internal comparator along with a composition without a polymeric additive as an internal negative control. Similar to the procedures described above, sodium and calcium lignosulfonates were individually added to microbial fermentates (20 g) for a final concentration of 4.0 wt % in each composition. Subsequent particle size analysis was performed on each sample at time 0 h and 24 h. Results from the particle size analysis are shown in FIG. 7 and FIG. 8.

TABLE 3

| Polymeric Additive | Product Type | Relative MW |
|---|---|---|
| POLYFON T | Sodium | 2900 |
| BORRESPERSE CA | Calcium | Medium |
| BORRESPERSE NA | Sodium | Medium |
| MARASPERSE AG | Sodium | Low |
| MARASPERSE CBOS-4 | Sodium | NA |
| MARASPERSE N-22 | Sodium | Medium |
| NORLIG 11D | Calcium | Low |
| UFOXANE 3A | Sodium | High |
| ULTRAZINE NA | Sodium | High |
| CONTROL | NA | NA |

### Example 5

In some instances, centrifugation with subsequent removal of the supernatant was performed on microbial compositions to increase the microbial titer. Experiments were performed to evaluate the effect of the timing of addition of the polymeric additive in relation to centrifugation.

In Experiment 5a, fermentation broth (30 g) was centrifuged using a THERMO SCIENTIFIC SORVALL LYNX 4000 SUPERSPEED centrifuge at 8000 rpm for 10 min. The supernatant (˜28-29 g) was removed followed by addition of 3 g of an aqueous solution of 4.0% POLYFON T. The composition was resuspended by vortex.

In Experiment 5b, fermentation broth (30 g) containing 4.0 wt % of POLYFON T was centrifuged at 8000 rpm for 10 min. The supernatant (˜28-29 g) was removed followed by addition of 3 g of deionized water and then the composition was resuspended by vortex.

In Experiment 5c, fermentation broth (30 g) containing 4.0 wt % POLYFON T was centrifuged at 8000 rpm for 10 min. The supernatant (˜28-29 g) was removed followed by addition of 3 g of a 4.0% aqueous solution of POLYFON T and then the composition was resuspended by vortex.

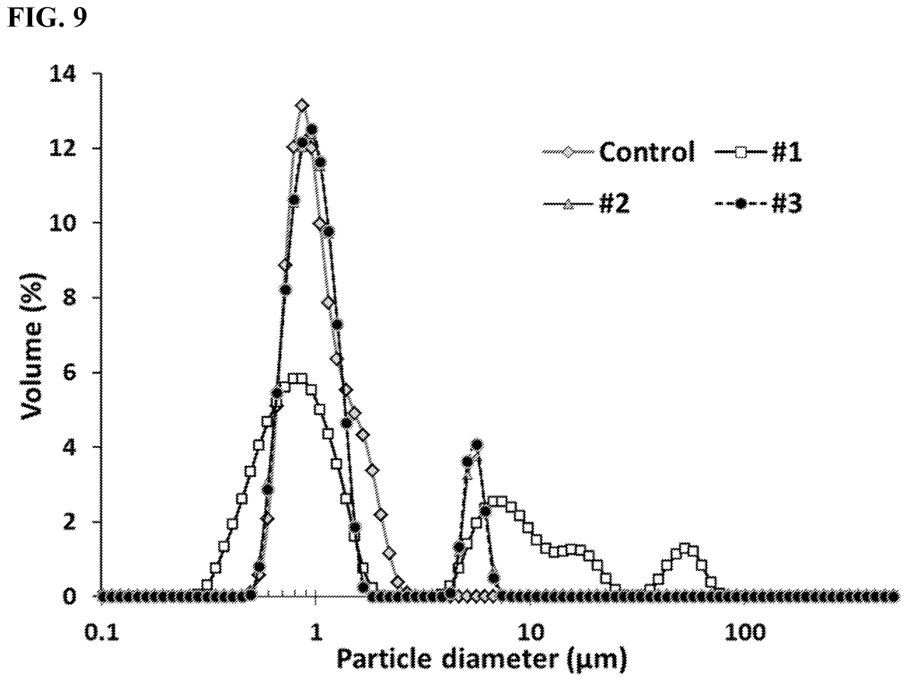

Subsequent particle size analysis was performed at 44 h on each sample. Results from the particle size analysis are shown in FIG. 9.

When introducing elements of the present invention or the preferred embodiments(s) thereof, the articles “a”, “an”, “the” and “said” are intended to mean that there are one or more of the elements. The terms “comprising”, “including” and “having” are intended to be inclusive and mean that there may be additional elements other than the listed elements.

In view of the above, it will be seen that the several objects of the invention are achieved and other advantageous results attained.

As various changes could be made in the above processes without departing from the scope of the invention, it is intended that all matter contained in the above description and shown in the accompanying drawings shall be interpreted as illustrative and not in a limiting sense.

1. A process for de-aggregating microbe spore aggregates, the process comprising:
mixing a polymeric additive with a first suspension comprising the microbe spore aggregates and one or more other components to form a second suspension comprising the polymeric additive, microbe spores and/or aggregates thereof, and the one or more other components, wherein the average particle size of the microbe spores and/or aggregates thereof in the second suspension is less than the average particle size of the microbe spore aggregates in the first suspension and wherein at least one of the following conditions is satisfied:
(a) the average particle size of the microbe spore aggregates in the first suspension is at least about 50 μm, at least about 60 μm, at least about 70 μm, at least about 80 μm, at least about 90 μm, at least about 100 μm, or at least about 200 μm;
(b) the average particle size of the microbe spores and/or aggregates thereof in the second suspension is less than about 50 μm, less than about 40 μm, less than about 30 μm, less than about 20 μm, less than about 10 μm, less than about 5 μm, or less than about 2 μm;
(c) the one or more other components comprises one or more microbe nutrients;
(d) the concentration of the polymeric additive in the second suspension is at least about 2 wt. %, at least about 2.5 wt. %, at least about 3 wt. %, at least about 3.5 wt. %, at least about 4 wt. %, at least about 4.25 wt. %, at least about 4.5 wt. %, at least about 5 wt. %, or at least about 6 wt. %; and/or
(e) the ratio of the microbe spores in CFUs to mass of the polymeric additive in grams in the second suspension is at least about 2×10⁵:1, at least about 2×10⁶:1, at least about 2×10⁷:1, at least about 2×10⁸:1, at least about 2×10⁹:1, or at least about 2×10¹⁰:1.

2. A process for producing a suspension concentrate comprising microbe spores and/or aggregates thereof, the process comprising:
mixing a polymeric additive with a first suspension comprising the microbe spore aggregates and one or more other components to form a second suspension comprising the polymeric additive, microbe spores and/or aggregates thereof, and the one or more other components, wherein the average particle size of the microbe spores and/or aggregates thereof in the second suspension is less than the average particle size of the microbe spore aggregates in the first suspension; and
separating at least a portion of the microbe spores and/or aggregates thereof from the one or more other components to form the suspension concentrate comprising the microbe spores and/or aggregates thereof.

3. The process of claim 2 wherein separating at least a portion of the microbe spores and/or aggregates thereof comprises centrifugation and/or filtration.

4. The process of claim 1 wherein the polymeric additive comprises a lignosulfonate.

5. The process of claim 4 wherein the lignosulfonate comprises a sodium lignosulfonate, a magnesium lignosulfonate, an ammonium lignosulfonate, a potassium lignosulfonate, a calcium lignosulfonate, or a combination thereof.

6. The process of claim 4 wherein the average molecular weight of the lignosulfonate is at least about 1,000 Da, at least about 2,000 Da, or at least about 2,500 Da.

7. (canceled)

8. The process of claim 4 wherein the lignosulfonate has a degree of sulfonation that is from about 0.3 moles/kg to about 4 moles/kg, from about 0.5 moles/kg to about 4 moles/kg, from about 0.5 moles/kg to about 3.5 moles/kg, from about 1 moles/kg to about 3.5 moles/kg, from about 1.2 moles/kg to about 3.3 moles/kg, or from about 1.2 moles/kg to about 2 moles/kg.

9. The process of claim 4 wherein the organic sulfur content of the lignosulfonate is from about 0.5 wt. % to about 20 wt. %, from about 1 wt. % to about 18 wt. %, from about 1 wt. % to about 16 wt. %, from about 2 wt. % to about 16 wt. %, from about 2 wt. % to about 10 wt. %, or from about 4 wt. % to about 10 wt. %.

10. The process of claim 1 wherein the polymeric additive comprises a maleic acid olefin copolymer.

11. The process of claim 1 wherein the concentration of the polymeric additive in the second suspension is at least about 2 wt. %, at least about 2.5 wt. %, at least about 3 wt. %, at least about 3.5 wt. %, at least about 4 wt. %, at least about 4.25 wt. %, at least about 4.5 wt. %, at least about 5 wt. %, or at least about 6 wt. %.

12. (canceled)

13. The process of claim 2 wherein the concentration of the microbe spores in the first suspension and/or second suspension is at least about 1×10⁵ CFUs, 1×10⁶ CFUs, at least about 1×10⁷ CFUs, at least about 1×10⁸ CFUs, at least about 1×10⁹ CFUs, at least about 1×10¹⁰ CFUs, or at least about 1×10¹¹ CFUs.

14. (canceled)

15. The process of claim 2 wherein the ratio of the microbe spores in CFUs to mass of the polymeric additive in grams in the second suspension is at least about 2×10⁵:1, at least about 2×10⁶:1, at least about 2×10⁷:1, at least about 2×10⁸:1, at least about 2×10⁹:1, or at least about 2×10¹⁰:1.

16. (canceled)

17. The process of claim 2 wherein the average particle size of the microbe spore aggregates in the first suspension is at least about 50 μm, at least about 60 μm, at least about 70 μm, at least about 80 μm, at least about 90 μm, at least about 100 μm, or at least about 200 μm.

18. (canceled)

19. The process of claim 1 wherein at least about 80 vol. %, at least about 85 vol. %, at least about 90 vol. %, at least about 95 vol. %, or at least about 97 vol. % of the microbe spore aggregates in the first suspension have a particle size greater than about 10 μm, greater than about 25 μm, greater than about 50 μm, greater than about 75 μm, or greater than about 100 μm.

20. The process of claim 2 wherein the average particle size of the microbe spores and/or aggregates thereof in the second suspension is less than 50 μm, less than about 40 μm, less than about 30 μm, less than about 20 μm, less than about 10 μm, less than about 5 μm, or less than about 2 μm.

21-23. (canceled)

24. The process of claim 1 wherein the pH of the second suspension is no greater than about 13, no greater than about 12.5, no greater than about 12, no greater than about 11.5, no greater than about 11, no greater than about 10.5, no greater than about 10, no greater than about 9.5, no greater than about 9, no greater than about 8.5, or no greater than about 8.

25-28. (canceled)

29. The process of claim 1 wherein the microbe spores are fungus spores, bacteria spores, or a combination thereof.

30. (canceled)

31. The process of claim 29 wherein the microbe spores comprise bacteria spores from at least one species selected from the group consisting of Bacillus amyloliquefaciens, Bacillus cereus, Bacillus firmus, Bacillus lichenformis, Bacillus psychrosaccharolyticus, Bacillus pumilus, Bacillus sphaericus, Bacillus subtilis, Bacillus thuringiensis, Pasteuria penetrans, Pasteuria usgae, and any combinations thereof.

32-33. (canceled)

34. A microbe composition comprising microbe spores and/or aggregates thereof and a polymeric additive, wherein at least one of the following conditions is satisfied:
(a) the average particle size of the microbe spores and/or aggregates thereof in the composition is less than about 50 μm, less than about 40 μm, less than about 30 μm, less than about 20 μm, less than about 10 μm, less than about 5 μm, or less than about 2 μm;
(b) the composition further comprises one or more other components;
(c) the concentration of the polymeric additive in the composition is at least about 2 wt. %, at least about 2.5 wt. %, at least about 3 wt. %, at least about 3.5 wt. %, at least about 4 wt. %, at least about 4.25 wt. %, at least about 4.5 wt. %, at least about 5 wt. %, or at least about 6 wt. %; and/or
(d) the ratio of the microbe spores in CFUs to mass of the polymeric additive in grams in the composition is at least about 2×10⁵:1, at least about 2×10⁶:1, at least about 2×10⁷:1, at least about 2×10⁸:1, at least about 2×10⁹:1, or at least about 2×10¹⁰:1.

35. The microbe composition of claim 34 wherein the polymeric additive comprises a lignosulfonate.

36. The microbe composition of claim 35 wherein the lignosulfonate comprises a sodium lignosulfonate, a magnesium lignosulfonate, an ammonium lignosulfonate, a potassium lignosulfonate, a calcium lignosulfonate, or a combination thereof.

37-63. (canceled)

64. The process of claim 2 wherein the polymeric additive comprises a lignosulfonate.

65. The process of claim 64 wherein the lignosulfonate comprises a sodium lignosulfonate, a magnesium lignosulfonate, an ammonium lignosulfonate, a potassium lignosulfonate, a calcium lignosulfonate, or a combination thereof.

66. The process of claim 2 wherein the polymeric additive comprises a maleic acid olefin copolymer.
